(19) United States
(12) Patent Application Publication
(10) Pub. No.: US 2007/0258969 A1
(43) Pub. Date: Nov. 8, 2007

(54) Compositions and methods for targeting metastatic tumors using multivalent ligand-linked carbohydrate polymers

(76) Inventors: Eliezer Zomer, Newton, MA (US); Anatole Klyosov, Newton, MA (US); David Platt, Newton, MA (US)

Correspondence Address: GREENBERG TRAURIG, LLP, MET LIFE BUILDING, 200 PARK AVENUE, NEW YORK, NY, 10166, US

(21) Appl. No.: 10/557,120

(22) PCT Filed: Mar. 18, 2005
(86) PCT No.: PCT/US05/09179

§ 371 Date: Oct. 30, 2006

Related U.S. Application Data
(60) Provisional application No. 60/554,848, filed on Mar. 19, 2004.

(51) Int. Cl. A61K 38/46; A61K 31/7072; A61K 31/704; A61K 31/7056; A61K 31/7048; A61K 31/737

(52) U.S. Cl. 424/94.6; 514/49; 514/27; 514/54; 514/34

(57) ABSTRACT

Disclosed herein are compositions comprising a drug linked to a multivalent ligand polysaccharide and the use of this composition in the prevention and treatment of cancer. In one aspect, the polysaccharide is obtained by physical and chemical treatment of naturally occurring polymers or semi synthetic polymers which are bridged specifically with one or more anti-cancer chemotherapeutic agents. Another aspect is directed to the effective delivery of the polysaccharide together with the chemotherapeutic agent to diseased tissue including cancerous tissue.

### FIELD OF THE INVENTION

The present invention is directed to the prevention and treatment of cancer. In particular, the current invention relates to a composition comprising a drug linked to a multivalent ligand polysaccharide.

### BACKGROUND OF THE INVENTION

It is well appreciated that the incidence of many forms of cancer is expected to increase as the population ages. For example, prostate cancer is the most commonly diagnosed cancer in American men as well as the second leading cause of male cancer deaths. Approximately 50% of patients diagnosed with prostate cancer have a form of the disease which has or will escape the prostate. Prostate cancer metastasizes to the skeletal system and patients typically die with overwhelming osseous metastatic disease. As yet, effective curative therapy is limited and very little palliative therapy is available for patients with metastatic disease.

It is known that the process of tumor cell metastasis requires that cells depart from the primary tumor, invade the basement membrane, traverse through the bloodstream from tumor cell emboli, interact with the vascular endothelium of the target organ, extravasate, and proliferate to form secondary tumor colonies.

It is generally accepted that many stages of the metastatic cascade involve cellular interactions mediated by cell surface components such as carbohydrate-binding proteins, which include galactoside binding lectins (galectins). For example, treatment of B16 melanoma and uv-2237 fibrosarcoma cells in vitro with anti-galectin monoclonal antibodies prior to their intravenous (i.v.) injection into the tail vein of syngenic mice resulted in a marked inhibition of tumor lung colony development. Transfection of low metastatic, low galectin-3 expressing uv-2237-c115 fibrosarcoma cells with galectin-3 cDNA resulted in an increase of the metastatic phenotype of the transfected cells. Furthermore, a correlation has been established between the level of galectin-3 expression in human papillary thyroid carcinoma and tumor stage of human colorectal and gastric carcinomas.

Simple sugars such as methyl-α-D-lactoside and lacto-N-tetrose have been shown to inhibit metastasis of B16 melanoma cells, while D-galactose and arabinogalactose inhibited liver metastasis of L-1 sarcoma cells.

Most common anticancer drugs are cytotoxic due to non-specific delivery to the tumor site. Clearly, there is a need for low toxic anticancer drugs. The present invention discloses a desirable therapeutic agent that facilitates target delivery. This target delivery enhances therapeutic efficiency of these anticancer drugs while substantially reducing their undesirable toxicity.

### BRIEF SUMMARY OF THE INVENTION

The present invention is directed to the prevention and treatment of cancer. In particular, the current invention relates to a composition comprising a drug linked to a multivalent ligand polysaccharide and the use of this composition in the prevention and treatment of cancer.

One embodiment of the present invention relates to a polysaccharide obtained by physical and chemical treatment of naturally occurring polymers or semi synthetic polymers that are bridged specifically with one or more anti-cancer chemotherapeutic agents. One aspect of the present embodiment is directed to effective delivery of the polysaccharide chemotherapeutic composition to diseased tissue including tumors.

Another embodiment of the present invention includes a polysaccharide as described above wherein the polysaccharide's backbone further includes galacturonic acid, and rhamnose is connected to a repeating unit of the polysaccharide. In one aspect, the polysaccharide further includes one or more neutral monosaccharides.

Another embodiment provides a negatively charged polysaccharide wherein at least one ligand side chain comprises one or more neutral saccharides connected to the backbone via a rhamnose unit in the backbone.

Another embodiment of the present invention includes a polysaccharide as described above wherein the polysaccharide backbone further includes mannose units and a carboxylic monosaccharide.

Another embodiment provides a negatively charged polysaccharide wherein at least one ligand side chain comprises one or more neutral saccharides connected to the backbone via a mannose unit in the backbone, wherein the anti-cancer agent is bridged to the carboxylic unit.

Another embodiment of the present invention includes a polysaccharide as described above wherein the polysaccharide backbone further includes a copolymer of glucuronate and glucosamine, a partially deacetylated hyaluronic acid derivative with molecular sizes ranging from about 2K to about 200K.

Another embodiment provides at least one ligand with a galactosyl terminal oligomer comprising one or more neutral saccharides connected to a backbone via an amino group of one or more glucosamine units in the backbone, wherein an anti-cancer agent is bridged to the carboxylic unit.

Another embodiment of the present invention includes a polysaccharide as described above wherein a polysaccharide backbone further includes glucosamine and N-acetylglucosamine polymer, and wherein the ligand unit has a galactose or rhamnose terminally bound through an amino group.

Another embodiment provides a positively charged polysaccharide wherein at least one ligand side chain comprises neutral saccharides connected to a backbone via an amino group in the backbone.

The present invention is also directed to a method of treating subjects diagnosed with cancer wherein a therapeutically effective amount of a chemically bridged anti-cancer drug linked to a polysaccharide is administered to a subject in need thereof. The chemically bridged composition can be administered by any acceptable route.

Another embodiment of the present invention is a method of preventing cancer in a subject post surgical intervention, radiation therapy or diagnosed as having a high risk of cancer, wherein a therapeutically effective amount of a linked polysaccharide with a chemically bridged anti-cancer drug is administered to a subject in need thereof.

Still another embodiment of the present invention is directed to a method for inhibiting metastasis in a subject wherein a therapeutically effective amount of an anti-cancer drug chemically-bridged to a linked-polysaccharide is administered to a subject in need thereof.

Other embodiments of the present invention include a pharmaceutical formulation for treating cancer wherein the formulation comprises an effective dose of an anti-cancer drug chemically-bridged to a linked-polysaccharide, wherein the polysaccharide has a backbone formed from a plurality of uronic acid saccharides and about one to ten monosaccharides connected to the backbone with terminal galactose, wherein an average total molecular weight is in the range of from about 2 kD to about 200 kD.

For a better understanding of the present invention, together with other and further objects thereof, reference is made to the accompanying drawings and detailed description and its scope will be pointed out in the appended claims.

### BRIEF DESCRIPTION OF THE SEVERAL VIEWS OF THE DRAWING

FIG. 1 is graph demonstrating cytotoxicity results using a carbohydrate-drug complex vs. the drug alone.

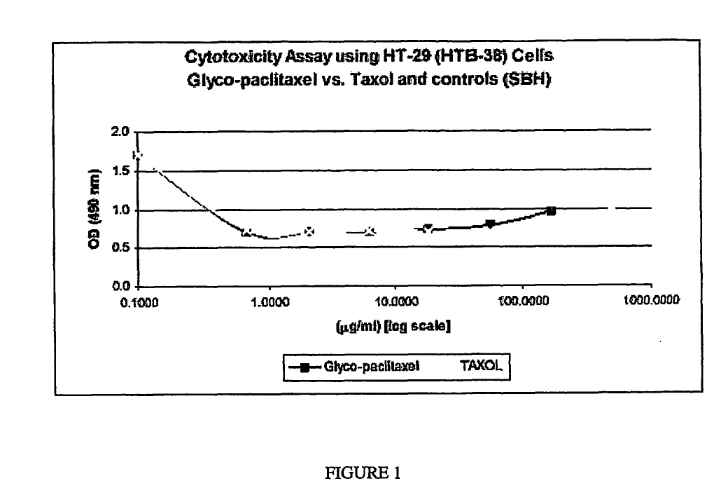

### DETAILED DESCRIPTION OF THE INVENTION

The present invention is directed to the prevention and treatment of cancer. In particular, the current invention relates to a composition comprising a drug linked to a multivalent ligand polysaccharide and the use of this composition in the prevention and treatment of cancer. In one aspect, the current invention relates to a polysaccharide obtained by physical and chemical treatment of naturally occurring polymers or semi synthetic polymers, which are bridged specifically with one or more anti-cancer chemotherapeutic agents. One aspect of the present invention is directed to the effective delivery of the polysaccharide together with the chemotherapeutic agents to diseased tissue including cancerous tissue.

Abbreviations used herein are: PS, polysaccharide; OS, oligosaccharide; EHS, Eaglebreth-Holm Swarm; DMEM, Dulbecco's Modified Eagle's Minimal Essential Medium; CMF-PBS, Ca²⁺- and Mg²⁺-Free Phosphate-Buffered Saline, pH 7.2; BSA, Bovine Serum Albumin; galUA, galactopyranosyl uronic acid, also called galacturonic acid; gal, galactose; man, mannose; glc, glucose; all, allose; alt, altrose; ido, idose; tal, talose; gul, gulose; ara, arabinose; rib, ribose; lyx, lyxose; xyl, xylose; fru, fructose; psi, psicose; sor, sorbose; tag, tagatose; rha, rhamnose; fuc, fucose; quin, quinovose; and 2-d-rib, 2-deoxy-ribose.

As used herein, the following terms shall have the meanings indicated, unless the context otherwise requires: “Administration” refers to parentereal including intravenous, subcutaneous, transdermal, transmucosal, intraperitoneal, and intramuscular or oral and topical.

“Subject” refers to an animal such as a mammal, e.g., a human.

“Treatment of cancer” refers to prognostic treatment of subjects at high risk of developing a cancer as well as subjects who have already developed a tumor. The term “treatment” may be applied to the reduction or prevention of abnormal cell proliferation, cell aggregation and cell dispersal (metastasis) to secondary sites.

“Cancer” refers to any neoplastic disorder, including, but not limited to, such cellular disorders as, renal cell cancer, Kaposi's sarcoma, chronic leukemia, breast cancer, sarcoma, ovarian carcinoma, rectal cancer, throat cancer, melanoma, colon cancer, bladder cancer, mastocytoma, lung cancer, mammary adenocarcinoma, pharyngeal squamous cell carcinoma, and gastrointestinal or stomach cancer.

“Anti-cancer drugs” chemicals that effectively hinder growth of proliferating cells including such molecules designated as cytotoxic, antimetabolite, anti-proliferation, anti-angiogenic, Antitumour Antibiotic, Alkylating Agent, Mitotic inhibitor, Endocrine Anti-hormone, Biological response modifier, tumor specific monoclonal antibody, apoptosis triggering agents and other molecules that effect cell viability.

“Depolymerization” refers to partial or complete hydrolytic degradation of the polysaccharide backbone occurring for example when the polysaccharide is treated chemically or enzymatically resulting in fragments of reduced size when compared with the original polysaccharide.

“Effective dose (or amount)” refers to a dose of an agent that improves the symptoms (and/or minimizes/eliminates the etiology for such symptoms) of the subject or the longevity of the subject suffering from or at high risk of suffering from cancer.

“Saccharide” refers to any simple carbohydrate including monosaccharides, monosaccharide derivatives, monosaccharide analogs, sugars, including those which form the individual units in an oligosaccharide or a polysaccharide.

“Monosaccharide” refers to polyhydroxyaldehyde (aldose) or polyhydroxy-ketone (ketose) and derivatives and analogs thereof.

“Oligosaccharide” refers to a linear or branched chain of monosaccharides that includes up to about 10 to 20 saccharide units linked via glycosidic bonds.

“Polysaccharide” refers to polymers formed from about 10 to about 10,000 and more saccharide units linked to each other by hemiacetal or glycosidic bonds. The polysaccharide may be either a straight chain, singly branched, or multiply branched wherein each branch may have additional secondary branches, and the monosaccharides may be standard D- or L-cyclic sugars in the pyranose (6-membered ring) or furanose (5-membered ring) forms such as D-fructose and D-galactose, respectively, or they may be cyclic sugar derivatives, for example, amino sugars such as D-glucosamine, deoxy sugars such as D-fucose or L-rhamnose, sugar phosphates such as D-ribose-5-phosphate, sugar acids such as D-galacturonic acid, or multi-derivatized sugars such as N-acetyl-D-glucosamine, N-acetylneurarninic acid (sialic acid), or N-sulfato-D-glucosamine.

“Backbone” means the major chain of a polysaccharide, or the chain originating from the major chain of a starting polysaccharide, having saccharide moieties sequentially linked by either α or β glycosidic bonds.

“Esterification” refers to the presence of methylesters or other ester groups at the carboxylic acid position of the uronic acid moieties of a saccharide.

“Substantially de-esterified” means, for the purposes of this application, that the degree of esterification on the backbone of the polysaccharide is less than about 5%.

“Substantially lacks secondary branches of saccharides” means that the polysaccharide backbone has less than about 1-2 secondary branches per repeating unit and no tertiary branches.

“Ligand” refers to a molecule that binds to another molecule, used especially to refer to a small molecule that binds specifically to a larger molecule, e.g., an antigen binding to an antibody, a hormone or neurotransmitter binding to a receptor, a substrate or allosteric effector binding to enzyme or receptor. For the purposes of this application, the carbohydrates that specifically bind to glyco-receptors on tumor cells are defied as “ligand”.

“Multivalent ligand binding polysaccharide” is a polysaccharide that possesses more than two or more ligand structures which will facilitate multiple binding sites per one polymer. Due to the multivalent receptor sites on tumor cells, the multivalent ligand binding will enable a stronger and more specific interaction between the polysaccharide and the tumor.

“Bridge” chemical structure which composes two or more molecules that chemically connects a specific agent (e.g., drug) to a delivery unit, for example, a polysaccharide polymer. For the purposes of this application the bridge is susceptible to degradation in a tumor micro-system. The Bridge could have a peptide structure made of 2 to 6 amino-acids, e.g., glycyl peptide, oligosaccharide with 2-6 carbohydrate, for example, an oligo (α1-4) glucosyl unit, any chemical with ester bonds or other bond that degraded in tumor cells.

“Glyco-receptors” refers to membrane-associated structures on cells exposed to the exterior of the cells and specifically bind carbohydrate molecules. Glyco-receptors refer to mainly proteins (including glycoproteins) associated with tumor cells and have been described in the literature as having high affinity to carbohydrate moieties, specifically the “galectins” which have high specific binding to galactose.

In accordance with the present invention, the polysaccharide component of the compositions described herein include a polymeric saccharide backbone comprising repeating units wherein each repeating unit can have a plurality of carboxylic acid which can be a free acid or alkylated, wherein each repeating sequence unit has at least one ligand comprising a mono or oligosaccharide residue attached thereto, at least one side chain of ligand oligosaccharide which is attached to the backbone via a glucosidic bond, and further comprising a plurality of polysaccharide derivatives with a majority of the ligand's terminating in, for example, a galactose unit. The polysaccharide includes a polymeric saccharide backbone comprising repeating units, wherein each repeating unit has a chemically bridged drug.

In one aspect, the polysaccharide's three dimensional structure has a unit structure where each unit contains at least one carbohydrate ligand and one chemically bridged drug (or pharmaceutical agent). The polysaccharide composition can have a plurality of such units with a total molecular weight ranging from about 2 kD for a single unit to about 200 kD for up to 1000 units, each with a bound anti-cancer agent. The composition is water soluble and targets metastatic cancer via interaction with galectins (glyco-receptors). Thus, the anticancer agents are targeted to cancer cells effectively and initiate less toxicity to normal tissue.

In one embodiment of the invention, a mulitivalent ligand polysaccharide has the capacity to deliver multiple units of the same pharmaceutical agent. Particular polysaccharides can have from about 1 to about 100 chemically bridged drugs, have up to 100 or greater ligands linked to the polysaccharide backbone, wherein each polysaccharide has a terminal saccharide comprising, for example, galactose, rharmnose, mannose, or derivatives thereof Other polysaccharides can have at least one side chain of saccharides ligand terminating with a saccharide modified by a feruloyl group.

In another embodiment of the invention, a multivalent-ligand-polysaccharide has the capacity to deliver multiple anti-cancer agents having diversified modes of action. Some polysaccharides can have up to 100 or greater chemically linked drugs, and have up to 100 or greater ligands linked to the polysaccharide back bone, each with a terminal saccharide comprising, for example, galactose, rhanmose, arabinose, or derivatives thereof. Other polysaccharides can have at least one side chain of saccharides ligand terminating with a saccharide modified by a feruloyl group.

Multiple variations of polysaccharides car be used in the present invention. In one aspect, the polysaccharides of the present invention further include a majority of galacturonic acid (optionally, including a neutral monosaccharide). In one aspect, rhamnose is connected to a repeating unit of a galacturonic acid-based polysaccharide. In another aspect, a negatively charged polysaccharide is employed wherein at least one ligand side chain comprises one or more neutral saccharides connected to the backbone via, for example, a rhamnose unit in the backbone. In a particular aspect, the polys accharides of the current invention include mannose units and one or more carboxylic monosaccharide. Another aspect is directed to a negatively charged polysaccharide having at least one ligand side chain that comprises one or more neutral saccharides connected to the backbone via a mannose unit in the backbone wherein the drug is linked to the carboxylic unit.

The polysaccharides of the present invention can include a copolymer of glucuronate and glucosamine, a partially deacetylated hyaluronic acid derivative having molecular sizes ranging from about 2K to about 200K.

Another embodiment provides at least one ligand with a galactosyl terminal oligomer comprising one or more neutral saccharides connected to a backbone via an amino group of one or more glucosamine units in the backbone, wherein the drug is bridged to the carboxylic unit.

Another embodiment of the present invention includes a polysaccharide as described above wherein a polysaccharide backbone further includes glucosamine and N-acetylglucosamine polymer, wherein the ligand unit has a galactose or rhamnose terminally bound through an amino group.

Another embodiment provides a positively charged polysaccharide wherein at least one ligand side chain comprises neutral saccharides connected to a backbone via an amino group in the backbone.

Other embodiments in accordance with the present invention include treating subjects diagnosed with cancer wherein a therapeutically effective amount of a chemically bridged drug linked to a polysaccharide is administered to a subject in need thereof.

Another embodiment of the present invention is a method of preventing cancer in a subject post surgical intervention, radiation therapy or diagnosed as having a high risk of cancer, wherein a therapeutically effective amount of a linked polysaccharide with a chemically bridged drug is administered to a subject in need thereof.

Still another embodiment of the present invention is a method for inhibiting metastasis in a subject wherein a therapeutically effective amount of a drug chemically-bridged to a linked-polysaccharide is administered to a subject in need thereof.

Other embodiments of the present invention include a pharmaceutical formulation for treating cancer wherein the formulation comprises an effective dose of a drug chemically-bridged to a polysaccharide, wherein the polysaccharide has a backbone formed from a plurality of uronic acid saccharides and about one to ten monosaccharides connected to the backbone, with terminal galactose and an average total molecular weight in the range of from about 2 kD to about 200 kD.

The composition comprising a polysaccharide conjugated to a drug of the present invention can be administered by any of several known routes including intravenous, subcutaneous, intraperitoneal, intramuscular, oral and topical routes, at equal intervals i.e., infusion from about 10 to about 2000 mg/kg over 2 to 24 hours and/or an injection of about 2.5 to 1000 mg/kg in 10 to 30 minutes.

The drugs or pharmaceutical agents of the present invention include, but are not limited to, anti-cancer drugs. Such anti-cancer drugs include, but are not limited to, aminoglutethimide, Amsacrine, Anastrozole, asparaginase, BCG, bicalutamide, Bleomycin, Buserelin, Busulfan, Capecitabine, carboplatin, Carmustine, chlorambucil, cisplatin, Cladribine, Clodronate, cyclophosphamide, cyproterone, Cytarabine, Dacarbazine, Dactinomycin, Daunorubicin, diethylstilbestrol, Docetaxel, Doxorubicin, Epirubicin, Estramustine, etoposide, Exemestane, Filgrastim, Fludarabine, Fludrocortisone, fluorouracil, Fluoxymesterone, Flutamide, Gemcitabine, Goserelin, hydroxyurea, Idarubicin, Ifosfamide, Imatinib, Interferon, Irinotecan, Letrozole, Leucovorin, Leuprolide, Levamisole, Lomustine, Mechlorethamine, Medroxyprogesterone, Megestrol, Melphalan, mercaptopurine, Mesna, methotrexate, mitomycin, Mitotane, Mitoxantrone, Nilutamide, Octreotide, Oxaliplatin, Paclitaxel, Pamidronate, Pentostatin, Plicamycin, Porfimer, procarbazine, Raltitrexed, Rituximab, streptozocin, Tamoxifen, Temozolomide, Teniposide, testosterone, thioguanine, Thiotepa, TNF-α, Topotecan, Trastuzumab, Tretinoin, Vinblastine, Vincristine, Vindesine, and Vinorelbine.

The compositions of the present invention can be formulated for oral or dermal administration, either alone or together with other excipients. Other routes of administration include intraperitoneal, intracranial, intracerebroventricular, intracerebral, intravaginal, intrauterine, rectal or parenteral (e.g., intravenous, intraspinal, subcutaneous or intramuscular) route. In addition, the present compositions can be incorporated into biodegradable polymers allowing for sustained release of the composition, the polymers being implanted in the vicinity of where drug delivery is desired, for example, at the site of a tumor or implanted so that the anti-cancer is slowly released systemically. Osmotic mini-pumps can also be used to provide controlled delivery of high concentrations of composition through cannulae to the site of interest, such as directly into a metastatic growth or into the vascular supply to that tumor, for example, see Brem et al., J. Neurosurg., (1991), vol. 74, pp. 441-446, the entire teaching of which is incorporated herein.

The effective dose and dosage regimen of the present compositions is a function of variables such as a subject's age, weight, medical history and other variables deemed to be relevant. The dose and dosage regimen based on the molecular weight of the present composition (i.e., disregarding the digestible carrier), and relative toxicity of the drug, may include a daily dose of about 10 to about 1000 mg per kg of body weight of the subject. The dosages of the present composition will depend on the disease state or condition being treated and other clinical factors such as weight and condition of the human or animal and the route of administration of the compound. Depending upon the half-life of the present composition in the particular animal, either or both agents can be administered between several times per day to once a week. It is to be understood that the present invention has application for both human and veterinary use. The methods of the present invention contemplate single as well as multiple administrations, given either simultaneously or over an extended period of time.

The present composition formulations include those suitable for oral, rectal, ophthalmnic (including intravitreal or intracameral), nasal, topical (including buccal and sublingual), intrauterine, vaginal or parenteral (including subcutaneous, intraperitoneal, intramuscular, intravenous, intradermal, intracranial, intratracheal, and epidural) administration. These formulations can conveniently be presented in unit dosage form and can be prepared by conventional pharmaceutical techniques. Such techniques include the step of bringing into association the active ingredient and a pharmaceutical carrier(s) or excipient(s). In general, the formulations are prepared by uniformly and intimately bringing into association the active ingredient with liquid carriers or finely divided solid carriers or both, and then, if necessary, shaping the product.

Formulations suitable for parenteral administration include aqueous and non-aqueous sterile injection solutions which can contain anti-oxidants, buffers, bacteriostats and solutes which render the formulation isotonic with the blood of the intended recipient; and aqueous and non-aqueous sterile suspensions which can include suspending agents and thickening agents. The formulations can be presented in unit- dose or multi-dose containers, for example, sealed ampules and vials, and can be stored in a freeze-dried (lyophilized) condition requiring only the addition of the sterile liquid carrier, for example, water for injections, immediately prior to use. Extemporaneous injection solutions and suspensions can be prepared from sterile powders, granules and tablets of the kdnd previously described.

In one aspect, unit dosage formulations are those containing a daily dose or unit, daily sub-dose, or an appropriate fraction thereof, of the administered ingredient. It should be understood that in addition to the ingredients, particularly mentioned above, the formulations of the present invention can include more than one agent as it is common in the field and have synergistic effect Optionally, anti-cancer agents can be incorporated or otherwise combined with the present composition to provide dual therapy to the patient.

For oral formulations a suitable digestible pharmaceutical carriers include gelatin capsules in which the present composition is encapsulated in dry form, or tablets in which polysaccharide is admixed with hydroxypropyl cellulose, hydroxypropyl methylcellulose, magnesium stearate, microcrystalline cellulose, propylene glycol, zinc stearate and titanium dioxide and other appropriate binding and additive agents. The composition can also be formulated as a liquid using distilled water, flavoring agents and some sort of sugar or sweetener as a digestible carrier to make a pleasant tasting composition when consumed by the subject.

An intestinal sustained-release matrix, as used herein, is a matrix made of materials, usually polymers, which are degradable by enzymatic or acid/base hydrolysis or by dissolution. Once introduced into the body, the matrix is acted upon by enzymes and body fluids. The sustained-release matrix desirably is chosen from biocompatible materials such as liposomes, polylactides (polylactic acid), polyglycolide (polymer of glycolic acid), polylactide co-glycolide (co-polymers of lactic acid and glycolic acid) polyanhydrides, poly(ortho)esters, polypeptides, hyaluronic acid, collagen, chondroitin sulfate, carboxylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. In one aspect, a biodegradable matrix is a matrix of one of either polylactide, polyglycolide, or polylactide co-glycolide (co-polymers of lactic acid and glycolic acid).

The metastasis-modulating therapeutic composition of the present invention can be a solid, liquid or aerosol and can be administered by any known route of administration. Examples of solid therapeutic compositions include pills, creams, and implantable dosage units. The pills can be administered orally; the therapeutic creams can be administered topically. The implantable dosage units can be administered locally, for example at a tumor site, or which can be implanted for systemic release of the therapeutic angiogenesis-modulating composition, for example, subcutaneously. Examples of liquid composition include formulations adapted for injection subcutaneously, intravenously, intra-arterially, and formulations for topical and intraocular administration. Examples of aerosol formulation include inhaler formulation for administration to the lungs.

Formulations suitable for parenteral administration include aqueous and non-aqueous sterile injection solutions which can contain anti-oxidants, buffers, bacteriostats and solutes which render the formulation isotonic with the blood of the intended recipient; and aqueous and non-aqueous sterile suspensions which can include suspending agents and thickening agents. The formulations can be presented in unit- dose or multi-dose containers, for example, sealed ampules and vials, and can be stored in a freezedried (lyophilized) condition requiring only the addition of the sterile liquid carrier, for example, water for injections, immediately prior to use. Extemporaneous injection solutions and suspensions can be prepared from sterile powders, granules and tablets of the kind previously described.

Typical unit dosage formulations are those containing a daily dose or unit, daily sub-dose, or an appropriate fraction thereof, of the administered ingredient. It should be understood that in addition to the ingredients, particularly mentioned above, the formulations of the present invention can include other agents conventional in the art having regard to the type of formulation in question. Optionally, cytotoxic agents can be incorporated or otherwise combined with angiostatin proteins, or biologically functional peptide fragments thereof, to provide dual therapy to the patient.

Other formulations for administering a therapeutic agent can be used that are well known in the art.

### EXAMPLES

### Example 1

### Bridging Tailored Sized Reduced Galacto-rhamnogalacturonic Polysaccharide to Paclitaxel

An anti-cancer drug can be chemically-bridged to a multivalent ligand-linked-polysaccharide by physically and chemically modifying naturally occurring polymers. Prior to treatment, the polysaccharides typically has a molecular weight ranging from about 40 kD to about 2000 kD or more with multiple branches of saccharides, e.g., branches comprised of glucose, arabinose, galactose, etc., (these branches can be connected to the backbone via neutral monosaccharides such as rhamnose). These molecules can further include a uronic acid saccharide backbone that can be esterified from as little as 10% to as much as about 90% (of uronic acid residues). The multiple branches themselves may have multiple branches of saccharides, the multiple branches optionally including neutral saccharides and neutral saccharide derivatives.

An outline of the procedure for physical and chemical treatment of naturally occuring rhamnogalactan involving a time-dependent temperature treatment at alkaline pH which specifically depolymerizes and de-branches acidic polysaccharide molecules is described herein. For example, the starting polysaccharide was treated at 37° C. for 30 minutes at pH 10.0. This was followed by cooling to room temperature. Following this initial cooling, the temperature was reduced to 4° C. and the pH was reduced to 3.2 in order to facilitate precipitation of a resulting polymer in a purer form. The preparation was subjected to stirring and elevated temperatures of about 55° C. for 1 to 24 hours resulting in the hydrolysis of the polymeric backbone and elimination of most, if not all, of the tertiary structures leaving primarily only the secondary branches with about 1 to about 10 carbohydrates, having a majority of galactose terminal groups.

An optional modification procedure involves the hydrolysis of a polysaccharide in an alkaline solution in the presence of a reducing agent such as potassium borohydride in order to form fragments of a repeating subunit (see U.S. Pat. No. 5,554,386, the entire teaching of which is incorporated herein by reference). The molecular weight target for the multivalent-ligand polysaccharide with chemically-bridged-drug is in the range of about 2 to about 200 kD, more specifically, in the range of about 15-40 kD, and even more specifically about 25 kD with a 1:5 ratio of anti-cancer drug to polysaccharide.

Typically, all of the manufacturing steps are conducted under good manufacturing practices. This is true for all of the examples disclosed herein. The amount of total carbohydrate was determined by the phenol sulfuric acid method (see Fidler et al., Cancer Res., (1973), vol. 41, pp. 3642-3956, the entire teaching of which is incorporated herein by references).

Paclitaxel is attached via an alkyl bridge with terminal amines via EDC cross linking as describe below.

The resulting polysaccharide-drug product was washed with 70% ethanol and dried from 95% ethanol. Thereupon the paclitaxel linked ligand polysaccharide was resolubilized in water to a final concentration of about 5% by weight (see Alberscheim et al., Carbohydrate Research, (1967) vol. 5, pp. 340-346, the entire teaching of which is incorporated herein by references).

The composition can be further diluted for use according to embodiments of the invention in which concentrations of 0.01-5% can be provided to cells. Depending on the raw starting material and the desired polysaccharide-drug composition (including the molecular weight), the reaction conditions can be further adjusted and modified accordingly.

(a) Late Coupling of Drug:

A glycopeptide was prepared through the conjugation of a commercially available tripeptide ester (H-Gly-Gly-Gly-OEt, Bachem, H-3350.0250) and Galacto-rhamnogalacturan (eRG) (60% esterified, 20 KDa) followed by deprotection and coupling with paclitaxel (2′-OH):

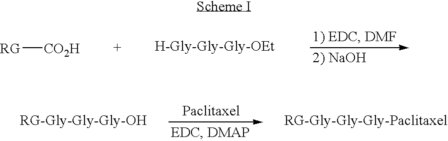

Paclitaxel is very expensive and toxic. Coupling it in the last step of the synthesis will reduce cost and facilitate handling of the conjugates.

The triglycine and similar peptide linkers have the advantages of being commercially available as ethyl ester and non-chiral (no racemization during couplings). Liquid chromatography with Evaporative Light Scattering Detection can be used for analysis of the glycopeptide. Altemative tripeptides as GlyPheGly esters may not be commercially available and would require in-house synthesis.

(b) Early Coupling of Drug:

Coupling of paclitaxel to Boc-Gly-Gly-Gly-OH (Bachem, A-4380.0005) using DIC/DMAP/DMF, deprotection of the terminal amino group with TFA/DCM and finally conjugation with eRG using EDC/DMF or water (depending on solubility) is depicted below.

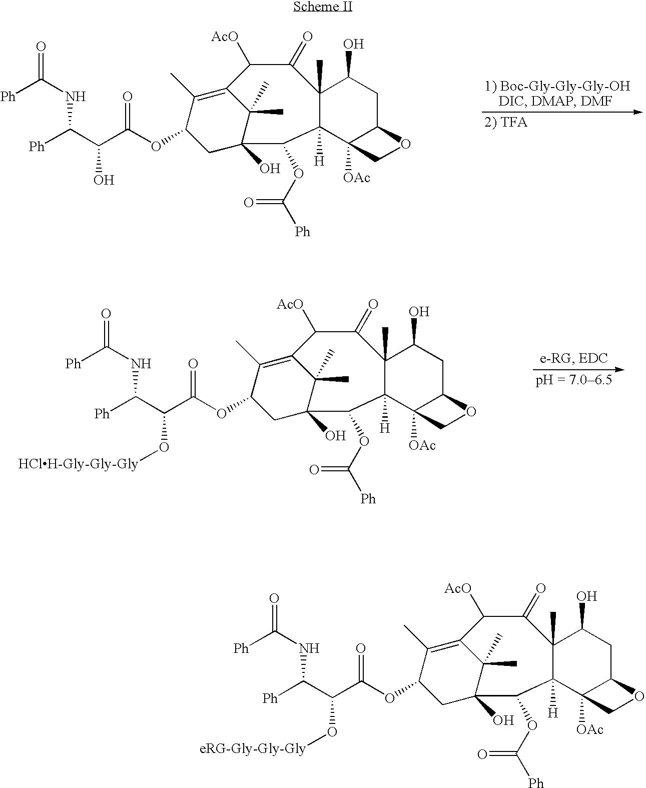

In this reaction scheme, paclitaxel is exclusively linked to eRG through the tripeptide linker.

Characterization and isolation of paclitaxel-tripeptide conjugate can be easier (HPLC, MS and NMR) than that of glycopeptide conjugate.

There are literature precedents for the deprotection of Boc-amino acids linked to taxol, e.g., U.S. Pat. No. 6,218,367, the entire teaching of which is incorporated herein by reference.

### Example 2

### Bridging Tailored Size Galacto-rhamnogalacturonic Polysaccharide to Paclitaxel through Hydrazinobenzoate Bridge

Described herein is an example of tailored size reduction, physical and chemical treatment of naturally occuring rhamnogalactan that involves time temperature treatment at alkaline pH which specifically depolymerizes and de-branches acidic polysaccharide molecules—for example, treatment at 37° C. for 30 minutes at pH titrated to pH 10.0, followed by cooling to room temperature. Further cooling to 4° C. and reduced pH to 3.2 to facilitate precipitation of the polymer in a purer form can be desirable. Additional treatment with high stiring and elevated temperature (to about 55° C. for about 1 to 24 hours) results in the hydrolysis of the polymeric backbone and elimination of the tertiary structures leaving only the secondary branches with about 1 to about 10 carbohydrates having a majority of galactose terminal groups.

An optional modification procedure involves the hydrolysis of the starting polysaccharide in an alkaline solution in the presence of a reducing agent such as a potassium borohydride to form fragments of a size corresponding to a repeating subunit (see, U.S. Pat. No. 5,554,386). The molecular weight target for the multivalent-ligand polysaccharide with chemically-bridged-drug is in the range of about 2 to about 200 kD, more specifically, in the range of about 15-40 kD, for example, about 25 kD, with a 1:5 ratio of anti-cancer drug to polysaccharide.

An example of paclitaxel linked ligand polysaccharide synthesis is as describe below.

(a) Hydrazinobenzoic Acid—Paclitaxel system

The following references can be consulted and are incorporated herein by reference:

Bull. Chem Soc. Jpn., 1981, 2219: Coupling of 4-hydrazinobenzoic acid with Boc-glutamic acid a-benzyl ester using DCC/Et3N/DCM has been shown to proceed in moderate yield (24% yield after ester and amino deprotection).

J. Org. Chem. 1979, 3752: Coupling ofeJhydrazoinobenzoic acid with N-benzyl-glutamic acid α-benzyl ester (ethyl chloroformate/triethylamine) gives the coupled product in reasonable yield (60% yield). The use of ethyl chloroformate tends to give better yields than carbodiimide. However, the free hydroxyl groups in the polysaccharide are not compatible with the use of ethyl chloroformate.

HydraLink (NovaBiochem): This technology for bioconjugation is based on the reaction of a 2-hydrazinopyridyl moiety and a benzaldehyde to yield a stable bis-aromatic hydrazone. The chemistry is highly selective and stable in solution. A terminal amino or hydroxyl group is necessary on each of the two fragments to conjugate.

Hydrazinobenzoic Acid Linker Strategy:

This strategy involves reacting RG with 4-hydrazinobenzoic acid followed by coupling with paclitaxel (2′-OH).

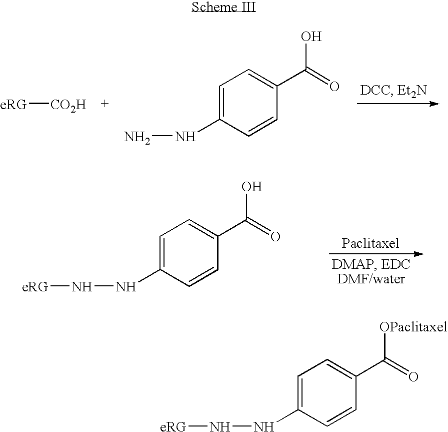

In this reaction, paclitaxel is conjugated on the last step of the synthesis reducing cost and facilitating intermediate handling.

The reagent, 4-hydrazinobenzoic acid, is inexpensive (Aldrich Chemicals) and does not required protection.

Hydrazinobenzoic Acid Linker Alternative Procedure:

An alternative strategy involves esterification of paclitaxel (2′-OH) with 4-(Fmoc-hydrazino)-benzoic acid (Bachem, Q-2545.001) followed by deprotection and conjugation with e-RG.

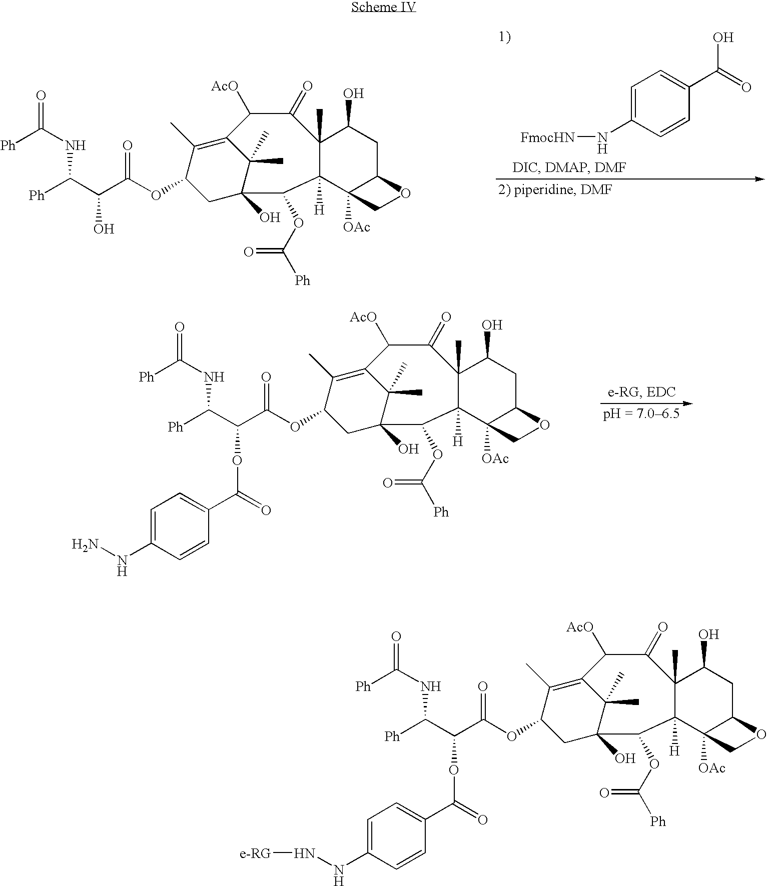

In this scheme, paclitaxel is exclusively linked to eRG through the hydrazinobenzoate linker.

### Example 3

### Bridging Tailored Size Galacto-rhamnogalacturonic Polysaccharide to Camptothecin

Described below is an example of tailored size reduction, physical and chemical treatment of naturally occuring rhamnogalactan that involves a time temperature treatment at alkaline pH which specifically depolymerizes and de-branches acidic polysaccharide molecules—for example, the material was subjected to treatment at 37° C. for 30 minutes at pH10, this was followed by cooling to room temperature. Further cooling to 4° C. and reducing pH to 3.2 was carried out in order to facilitate precipitation of the polymer in a substantially isolated form. The preparation was further treated by raising the temperature to 55° C. for 1 to 24 hours resulting in the hydrolysis of the polymeric backbone and elimination of the tertiary structures essentially leaving only the secondary branches with 1 to 10 carbohydrate having a majority of galactose terminal groups.

An optional modification procedure involves the hydrolysis of the polysaccharide in an alkaline solution in the presence of a reducing agent such as a potassium borohydride which results in fragments of a size corresponding to a repeating subunit (see U.S. Pat. No. 5,554,386). The molecular weight target for the multivalent-ligand polysaccharide with chemically-bridged-drug is in the range of about 2 to about 200 kD, more specifically, in the range of about 15-40 kD, for example, 25 kD, with a 1:5 ratio of anti-cancer drug to polysaccharide.

To prepare camptothecin conjugates, a process similar to Poly(L-glutamic acid) (PG) conjugates of 20-S-Camptothecin was employed. The direct esterification of 20-hydroxyl group in Camptothecin with PG using BOP-CV DMAP/DMF gave a low yield due to steric constrains of the hydroxyl group. However, acylation of camptothecin with Boc-Gly-Gly-Gly follow by deprotection provided better results (DIC/DMAP/DMF, then TFA/DCM). Finally, the cytotoxic-peptide conjugate was employed as a TFA salt for the coupling with poly (L-glutamic acid) (3eq DMAP/DIC/DMF). (See, J. Med. Chem. (2003) 46, 190-193, the entire teaching of which is incorporated herein by reference.)

Conjugates of 20-S-Camptothecin+peptide+carbohydrate.

Valine-20-O-Camptothecin was prepared using Boc-valine-N-carboxyanhydride (not commercially available) and DMAP followed by coupling of subsequent amino acids to valine-cytotoxic conjugate using EDC/HOBt/DMF. (See, U.S. Pat. Nos. 6,506,734 and 6,492,335, the entire teachings of which are incorporated herein by reference.)

Conjugates of 20-S-Camptothecin+PEG40kD+20-S-Camptothecin

Coupling of PEG40D-dicarboxyhc acid with 20-S-Camptothecin using DIC/DMAP/DCM gave a mixture of mono and diester (1.35 eq/PEG). (See, U.S. Pat. No. 5,880,131.)

Coupling of PEG-glycine with 20-S-Camptothecin under the same conditions gave the corresponding dimer in 69% yield.

20-Bromoacetyl-camptothecin was prepared by reacting bromoacetic acid (67% yield) and with PEG-dicarboxylic acid to obtain 1.6 eq of camptothecin per PEG molecule (76% yield).

Another conjugate having similar chemistry to camptothecin is 5-deoxy-5-fluorocytidine conjugated to carboxylated galactomannan and/or rhamnogalacturonan. This conjugate can generate the pro-drug 5-DFCR in, for example, the intestine or blood converting it to 5-fluorouracil.

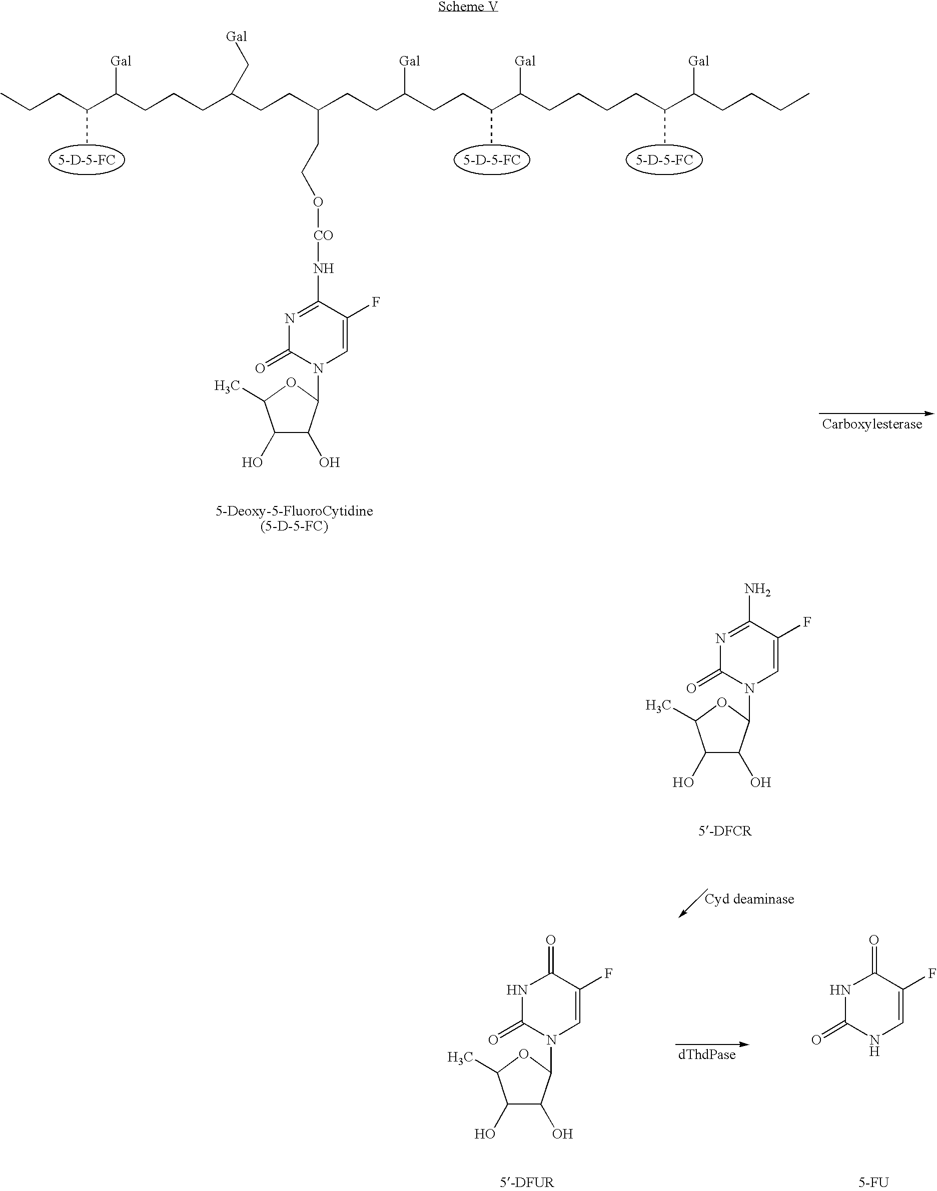

Tripeptide Linker Composition:

The following procedure can be used to produce a tripeptide linker composition. Prepare a glycopeptide through conjugation of a commercially available tripeptide ester (e.g., Gly-Gly-Gly-OEt, Bachem, H-3350.0250) with galacto-rhamnogalacturan (eRG) (60% esterified, 20 KDa) followed by deprotection and coupling with camptothecin (20-OH). (Same approach as described in Example 1.)

The steric constrains of the 20-hydroxyl group in camptothecin coupled to the glycopeptide, eRG-Gly-Gly-Gly-H, could lead to low loading of the drug. This can be avoided by coupling first camptothecin to BocGlyGlyGly (Bachem, A4380.0005) using DIC/DMAP/DMF followed by TFA/DCM. This is followed by preparing the conjugate with RG using EDC or DIC. (Similar approach as in Example 1.)

### Example 4

### Bridging Tailored Sized Galacto-rhamnogalacturonic Polysaccharide to Camptothecin

Below is described an example of tailored size reduction, physical and chemical treatment of naturally occuring rhamnogalactan that involves a time temperature treatment at alkaline pH which depolymerizes and de-branches acidic polysaccharide molecules. The reaction conditions were essentially 37° C. for 30 minutes at pH 10.0. This was followed by cooling to room temperature, followed by further cooling to 4° C. and reducing pH to 3.2 in order to facilitate precipitation of a polymer into an essentially isolated form The preparation was further subjected to an elevated temperature of around 55° C. for 1 to 24 hours resulting in the hydrolysis of the polymeric backbone and elimination of the tertiary structures leaving only the secondary branches with about 1 to 10 carbohydrate having a majority of galactose terminal groups.

An optional modification procedure was the hydrolysis of the polysaccharide using an alkaline solution in the presence of a reducing agent such as a potassium borohydride in order to form fragments of a size corresponding to a repeating subunit (see U.S. Pat. No. 5,554,386). The molecular weight target for the multivalent-ligand polysaccharide with chemically-bridged-drug is in the range of about 2 to about 200 kD, more specifically, in the range of about 15-40 kD, for example, 25 kD, with 1:5 ratio of anti-cancer drug to polysaccharide.

(10)-Hydroxy Camptothecin:

Similar to that disclosed in Cancer Research (2000) 2988 and U.S. Pat. No. 5,837,673, the entire teachings of which are incorporated herein by reference, conjugates of 10-hydroxy camptothecin derivative were manufactured. First, the synthesis of a 10-hydroxy camptothecin derivative using 3-aminopropyl ether at carbon-10 was performed. A tri or larger peptide was attached to this amino group using Boc as the protecting group. Coupling to a ligand polysaccharide was accomplished with EDC/water at pH=7.0-6.5 to the carboxylic acid in one step.

### Example 5

### Bridging Tailored Sized Galacto-rhamnogalacturonic Polysaccharide to TNF-α

Described below is an example of tailored size reduction, physical and chemical treatment of naturally occuring rhamnogalacan that involve a time temperature treatment at alkaline pH which specifically depolymerizes and de-branches acidic polysaccharide molecules. The starting material was subjected to 37° C. for 30 minutes at pH 10.0. This was followed by cooling to room temperature. Further cooling to 4° C. and reducing the pH to 3.2 in order to facilitate precipitation of the polymer was performed. The preparation was next subjected to elevated temperatures around 55° C. for 1 to 24 hours resulting in the hydrolysis of the polymeric backbone and elimination of the tertiary structures leaving only the secondary branches with 1 to 10 carbohydrate having a majority of galactose terminal groups.

An alternative modification procedure involves the hydrolysis of the polysaccharide using an alkaline solution in the presence of a reducing agent such as a potassium borohydride in order to form fragments of a size corresponding to a repeating subunit (see U.S. Pat. No. 5,554,386). The molecular weight target for the multivalent-ligand polysaccharide with chemically-bridged-drug is in the range of about 2 to about 200 kD, more specifically, in the range of about 15-40 kD, for example, 25 kD, with 1:5 ratio of anti-cancer drug to polysaccharide. The binding to TNF-A is accomplished with any water soluble cross linker such as EDC (Pierce).

### Example 6

### Demonstrating the Biological Efficacy of Administered Multivalent-ligand Polysaccharide with Chemically-bridged Anti-cancer Drugs using In vitro and In vivo Assays

To show the efficacy of the multivalent-ligand-polysaccharide (mLP) with chemically-bridged-anti-cancer drugs (bDRUG), the inventors selected a number of in vitro and in vivo assays to demonstrate the biological efficacy of the mLP-bDRUG. Inhibition of metastasis can be shown using cancer cell lines which normally aggregate in culture. In the presence of mLP-bDRUG, the tumor cells (e.g., colon TH-29, melanoma B16-F1) remained dispersed and slow death was observed. Cell lines such as B16-F1 cell, UV 2237-10-3 murine fibrosarcoma cells, HT 1080 human fibrosarcoma cells, and A375 human melanoma cells can be employed for these assays. Inhibition and death of metastasis can also be demonstrated using a metastasis assay in which MLL cells which have enhanced levels of galectins-3 on their cell surface, which appears to be associated with tumor endothelial cell adhesion.

Vertebrate galactoside-binding lectins occur in a variety of tissues and cells. The lectins are divided into two abundant classes based on their size: molecular masses of about 14 kD and about 30 kD are designated as galectin-1 and galectin-3, respectively. Galectin-3 represents a wide range of molecules i.e., the murine 34 kD (mL-34) and human 31 kD (hL-31) tumor-associated galactoside-binding lectins, the 35 kD fibroblast carbohydrate-binding protein (CBP35), the IgE-binding protein (cBP), the 32 kD macrophage non-integrin laminin-binding protein (Mac-2), and the rat, mouse, and human forms of the 29 kD galactoside-binding lectin (L-29). Molecular cloning studies have revealed that polypeptides of these lectins share identical amino acid sequences.

Galectin-3 is highly expressed by activated macrophages and oncogenically transform into metastatic cells. Many cancer cells, including MLL cells, express galectin-3 on their cell surface and its expression has been implicated in metastatic processes in tumor cells. Elevated expression of the polypeptide is associated with an increased capacity for anchorage-independent growth, homotypic aggregation and tumor cell lung colonization, suggesting that galectin-3 promotes tumor cell embolization in the circulation and enhances metastasis. Tumor-endothelial cell adhesion is thought to be a key event in the metastatic process. Galectins bind with high affinity to oligosaccharides containing poly-N-acetyllactosamine sequences and also bind to the carbohydrate side chains of laminin in a specific sugar-dependent manner. Laminin, the major non-collagenous component of basement membranes, is an N-linked glycoprotein carrying poly-N-acetyllactosamine sequences and is implicated in cell adhesion, migration, growth, differentiation, invasion and metastasis.

Tumor cells can interact with carbohydrate residues of glycoproteins via cell surface galectin-3 and this can be correlated with their ability to interact with the galactose residues of agarose (a polymer of D-galactose and L-anhydro-galactose) and to provide the minimal support needed for cell proliferation in this semi-solid medium. Anti-galectin-3 monoclonal antibodies can inhibit the growth of tumor cells in agarose. Furthermore, transfection of normal mouse fibroblasts with the mouse galectin-3 cDNA results in the acquisition of anchorage-independent growth. The in vivo results reported here with polysaccharides are consistent with studies reported earlier and performed on human prostate cancer tissue using galectin-3 (U.S. Pat. No. 5,895,784, the entire teaching of which is incorporated herein by reference).

(a) Administration of Polysaccharide and Anti-cancer Drug to Animals with Tumors

(i) In vitro

A polysaccharide preparation was administered with a chemically bridged cancer drug at a therapeutic dose of the anti-cancer drug known in the art to be effective against cancer. As one example, camptothecin-mLP was administered at 1-10 mg/mL equivalents at doses of 30 to 300 mg/m2/day. The results in tumor bearing animals indicate that, for example, camptothecin as described above, are chemically linked at ratio of 1 to 5 (mole/mole) of a polysaccharide at doses of 60 to 600 mg/m². In particular, it was observed for rodents with colon tumors that up to 80% demonstrated improvement in toxicity with mLP-camptothecin versus the un-bound drug.
(ii) In vitro

Assays for determining anti-cancer efficacy of paclitaxel bridged—mLP were carried out using standard 96 wells plates. Unless stated otherwise, the assays below were conducted using a mLP-paclitaxel as described above. Controls include (i) polysaccharide alone, (ii) gal-, rha- or man-substituted saccharide; or (iii) anti-cancer drug alone. The results with human colon cancer HT-29 indicate that the mLP bridged paclitaxel is active in the microgram per mL concentration and equivalent to the common formulation of paclitaxel.

Laminin and Asialoglycoprotein Adhesion Assays:

This assay was used to establish the correlation between the propensity of tumor cells to undergo homotypic aggregation in vitro and their metastatic potential in vivo. B16 melanoma cell clumps produce more lung colonies after i.v. injection than do single cells. Moreover, anti-galectin-3 antibody has been shown to inhibit asialofetuin-induced homotypic aggregation (Fidler, I. J., (1970) J. Natl. Cancer Inst., 45:77, the entire teaching of which is incorporated herein by reference), suggesting that the cell surface galectin-3 polypeptides bring about the formation of homotypic aggregates following their interaction with the side chains of glycoproteins.

A mLP paclitaxel made according to this Example was tested for its ability to control cell-cell and cell-matrix interactions in B16-F1 murine melanoma cell adhesion assays which include measuring a change in adhesion of cells to a laminin coated substrate and inhibition of asialofetuin-induced homotypic aggregation and cell growth.

The B16-F1 line (low incidence of lung colonization) are derived from pulmonary metastasis produced by intravenous injection of B16 melanoma cells (Lotan, R. et al., Int. J. Cancer, (1994), vol. 56, pp. 1-20, the entire teaching of which is incorporated herein by reference). Other cell lines that can be tested include UV-2237-10-3 Murine Fibrosarcoma Cells, HT 1080 Human Fibrosarcoma Cells, and A375 Human Melanoma Cells.

The cells were grown in a monolayer on plastic in Dulbecco's modified Eagle's minimal essential medium (DMEM) supplemented with glutamine, essential amino acids, vitamins, antibiotics, and 10% heat-inactivated fetal bovine serum (FCS 10%). The cells are maintained at 370 C in a humidified atmosphere of 7% CO2, 93% air. To ensure reproducibility, all experiments should be performed with cultures grown for no longer than six weeks after recovery of stocks.

Cell Adhesion to Laminin

Tissue culture wells of 96-well plates were pre-coated overnight at 4° C. with EHS laminin (2 mg/well) in Ca2+- and Mg2+-free phosphate-buffered saline, pH 7.2 (CMF-PBS), and the remaining protein binding sites were blocked for 2 h at room temperature with 1% bovine serum albumin (BSA) in CMF-PBS. Cells were harvested with 0.02% EDTA in CMP-PBS and suspended with serum-free DMEM. A total of 5×104 cells were added to each well in DMEM with or without: 1) paclitaxel-mLP of varying concentrations 2) ligand polysaccharide of varying concentrations or 3) paclitaxel in common formulation of varying concentrations. After incubation for 2 h to 4 hr at 37° C., non-adherent cells were washed off with CMF-PBS and adherent cells were fixed with methanol and photographed. The relative number of adherent cells was determined in accordance with the procedure of Zollner, T. et al., Anti-cancer Research, (1993), vol. 13, pp. 923-930, the entire teaching of which is incorporated herein by reference. Briefly, cells were stained using methylene blue followed by the addition of HCl-ethanol to release the dye. The optical density (650 hm) was then measured by a plate reader.

MLP-bDRUG Binding to Lectins

Binding to lectins has been correlated with membrane surface changes with changes in membrane permeability. Recombinant galectin-3 can be extracted from bacteria cells by a single-step purification through an asialofetuin affinity column as known to those skilled in the art. For example, recombinant galectin-3 eluted by lactose was extensively dialyzed against CMF-PBS before use. Horseradish peroxidase (HRP)- conjugated rabbit anti-rat IgG+IgM and the 2, 2′-azino-di(3-ethylbenzthiazoline sulfonic acid) (ABTS) substrate kit can be purchased from Zymed, South San Francisco, Calif. B16-F1 murine melanoma cells were grown as cultures in Dulbecco's modified Eagles' minimal essential medium (DMEM, as described above.

Tissue culture wells of 96-well plates were coated with CMF-PBS containing 0.5% mLP and 1% BSA and dried overnight. Recombinant galectin-3 serially diluted in CMF-PBS containing 0.5% BSA and 0.05% Tween-20 (solution A) in the presence or absence of 50 mM lactose was added and incubated for 120 minutes, after which the wells were drained and washed with CMF-PBS containing 0.1% BSA and 0.05% Tveen-20 (solution B). Rat antigalectin-3 in solution A was added and incubated for 60 minutes, followed by washing with solution B and incubation with HRP-conjugated rabbit anti-rat IgG+IgM in solution A for 30 minutes. After washing, relative amounts of bound enzyme conjugated in each well were ascertained by addition of ABTS. The extent of hydrolysis was measured at 405 hn.

Colony Formation in Semi-solid Medium

Cells were detached with 0.02% EDTA in CMF-PBS and suspended at 1×103 cell/mL in complete DMEM with or without: 1) cytotoxic drug linked to multivalent ligand polysaccharide of varying concentrations, 2) multivalent ligand polysaccharide of varying concentrations, or 3) Cytotoxic drug alone. The cells were incubated for 30 min at 37° C. and then mixed 1:1 (v/v) with a solution of 1% agarose in distilled water-complete DMEM (1:4, v/v) preheated at 45° C. Next, 2 mL aliquots of the mixture were placed on top of a pre-cast layer of 1% agarose in 6-cm diameter dishes. The cells were incubated for 14 days at 37° C. and the number of formed colonies was determined using an inverted phase microscope after fixation by the addition of 2.6% gluteraldehyde in CMF-PBS.

Galectin-3 Heterotypic aggregation Metastasis Assay

The MAT-LyLu (MLL) sub-line is a fast growing, poorly differentiated adenocarcinoma cell line. The adhesion of Cr-labeled MLL cells to confluent monolayers of rat aortic endothelial (RAE) cells in the presence or absence of cytotoxin linked ligand polysaccharide was investigated. First, MLL and RAE cells were grown in RPMI 1640 media supplemented with 10% fetal bovine serum. RAE cells were grown to confluence in tissue culture wells. A total of 2.4×106 MLL cells were incubated for 30 minutes with 5 mCi Na5CrO4 at 37° C. in 2 mL of serum-free media with 0.5% bovine serum albumin (BSA). Following extensive washing, 1×103 MLL cells per well were added to RAE cell monolayers in quadruplicate. Attachment of MLL cells in the absence or presence of independently varied concentrations of combined cytotoxin linked ligand polysaccharide and anti-cancer drug for 90 minutes at 4° C. was then assessed as follows. The cells were washed three times in cold phosphate-buffered saline to remove unbound cells and then solubilized with 0.1 N NaOH for 30 minutes at 37° C. at which point the radioactivity in each well is determined in a β-counter. The time course for the attachment of MLL cells to a confluent monolayer of RAE cells in the absence or presence of independently varied concentrations of cytotoxin linked ligand polysaccharide in chemically bridged with an anti-cancer drug is monitored. The level of cytotoxin linked ligand polysaccharide /anti-cancer drug inhibition on attachment of MLL cells to RAE cells was thereby determined.

Alternatively, MLL cell adhesion to RAE cells was monitored through fluorescence methods. First, 1×105 MLL cells were incubated for 30 minutes in 0.1% FITC to fluorescently label the cells. Following extensive washing the cells were added to RAE cell monolayers in 0.5% BSA. Independently varying concentrations of mLP-bDRUG were added for 30 or 60 minutes. The cultures were then washed to remove non-adherent cells and the level of adhesion, or non-adhesion, was assessed based on fluorescence measurements.

To address the binding of mLP-bDRUG to galectin-3, an enzyme-linked immunosorbent assay was employed to determine whether recombinant galectin-3 is able to bind immobilized cytotoxic drug chemically linked to ligand polysaccharide in a dose-dependent manner and whether the binding, if it occurs, is capable of being blocked by lactose. Results from such an assay allow assessment of the inhibitory effects on homotypic aggregation of a mLP-bDRUG administered in chemically bridged with an anti-cancer drug, in accordance with the present invention, and determine whether any mLP-bDRUG binding occurs via cell surface galectin-3 molecules.

In vivo Assays for Determining Efficacy of mLP-bDRUG

(a) Inhibition of Metastasis of R3327-MLL Cells In Vivo

The Dunning (R3327) rat prostate adenocarcinoma model of prostate cancer was developed from a spontaneously occurring adenocarcinoma found in a male rat as described by Dunning, W., Natl. Cancer Inst, Mono., (1963), vol. 12, pp. 351-369, the entire teaching of which is incorporated herein by reference. Several sub-lines have been developed from the primary tumor which possess varying differentiation and metastatic properties as described by Isaacs, J. et al., Cancer Res., (1978), vol. 38, pp. 4353-4359, the entire teaching of which is incorporated herein by reference. Injection of 1×106 MLL cells into the thigh of the rat leads to animal death within approximately 25 days secondary to overwhelming primary tumor burden as described by Isaacs, J. et al. (Also, see, The Prostate, (1986), vol. 9, pp. 261-281, and Pienta, K., et al., The Prostate, (1992), vol 20, pp. 233-241, the entire teachings of which are incorporated herein by reference. The primary MLL tumor starts to metastasize approximately 12 days after tumor cell inoculation and removal of the primary tumor by limb amputation prior to this time results in animal cure. If amputation is performed after day 12, most of the animals die of lung and lymph node metastases within 40 days as described by Isaacs, J. et al., The Prostate, (1986), vol. 9, pp. 261-281.

mLP-bDRUG is given orally to rats in the drinking water on a chronic basis, to investigate the affect on spontaneous metastases is these tumors. The rats were first injected with 1×106 MTT cells in the hind limb on day 0. On day 4, when the primary tumors reach approximately 0.1 cm3 in size, 0.01%, 0.1%, or 1.0% (w:v) paclitaxel conjugated to ligand polysaccharide was added to the drinking water of the rats on a continuous basis. On day 14, the rats were anesthetized and the primary tumors removed by amputating the hind limb. The rats were then followed to day 30 when all groups were sacrificed and autopsied. Animals continuously ingest cytotoxin linked ligand polysaccharide/anti-cancer drug in their drinking water during this period. Control and treated animals are monitored for observable toxicity.

At day 30, the lungs were removed, rinsed in water and fixed overnight in Bouin's Solution. The number of rats which suffer lung metastases were compared to those in the control and recorded. The number of MLL tumor colonies was determined by counting under a dissection microscope.

Control and co-treated animals gained weight appropriately and no observable additional toxicity in the paclitaxel conjugated to ligand polysaccharide treated animals was observed when compared to a control treatment of anti-cancer drug alone. The number of rats which suffer lung metastases was reduced in animals fed with drug bridged to multivalent ligand polysaccharide of the type described above. A similar pattern of effect can be observed for lymph node disease. This example treatment was designed to show an improved method of treating an animal using non-toxic orally administered cytotoxic drug when linked to ligand to prevent spontaneous cancer metastasis.

Although the invention has been described with respect to various embodiments, it should be realized this invention is also capable of a wide variety of futher and other embodiments within the spirit and scope of the appended claims.

What is claimed is:

1. A composition, comprising a polysaccharide and one or more chemotherapeutic agents, wherein said chemotherapeutic agents are chemically linked to said polysaccharide.

2. The composition of claim 1, wherein said polysaccharide has from about 10 to about 10,000 saccharide units.

3. The composition of claim 1, wherein said one or more chemotherapeutic agents linked to polysaccharide ranges from 1 to about 100 chemically bridged agents.

4. The composition of claim 2, wherein said saccharide units are linked to each other by a hemiacetal or glycosidic bond.

5. The composition of claim 4, wherein said units have a molecular weight ranging from about 2 kD to about 1000 kD.

6. The composition of claim 2, wherein said saccharide units are cyclic sugars selected from the group consisting of D-cyclic sugars, L-cyclic sugars, or derivatives thereof.

7. The composition of claim 6, wherein said cyclic sugars are either pyranose or furanose sugars.

8. The composition of claim 6, wherein said cyclic sugars are selected from the group consisting of fructose, galactose, glucosamine, fucose, fhamnose, ribose-5-phosphate, galacturonic acid, N-acetyl-D-glucosamine, N-acetylneurminic acid, or N-sulfato-D-glucosamine.

9. The composition of claim 1, wherein said polysaccharide is either a straight chain, singly branched, or multiply branched, wherein each branch can have additional secondary branches.

10. The composition of claim 1, wherein said chemotherapeutic agent is an anti-cancer agent.

11. The composition of claim 9, wherein said anticancer agent is selected from the group consisting of aminoglutethimide, Amsacrine, Anastrozole, asparaginase, BCG, bicalutamide, Bleomycin, Buserelin, Busulfan, Capecitabine, carboplatin, camptothecin, Carmustine, chlorambucil, cisplatin, Cladribine, Clodronate, cyclophosphamide, cyproterone, Cytarabine, Dacarbazine, Dactinomycin, Daunorubicin, diethylstilbestrol, Docetaxel, Doxorubicin, Epirubicin, Estustine, etoposide, Exemestane, Filgrastim, Fludarabine, Fludrocortisone, fluorouracil, Fluoxymesterone, Flutamide, Gemcitabine, Goserelin, hydroxyurea, Idarubicin, Ifosfamide, Imatinib, Interferon, Irinotecan, Letrozole, Leucovorin, Leuprolide, Levamisole, Lomustine, Mechlorethamine, Medroxyprogesterone, Megestrol, Melphalan, mercaptopurine, Mesna, methotrexate, mitomycin, Mitotane, Mitoxantrone, Nilutamide, Octreotide, Oxaliplatin, Paclitaxel, Pamidronate, Pentostatin, Plicamycin, Porfimer, procarbazine, Raltitrexed, Rituximab, streptozocin, Tamoxifen, Temozolomide, Teniposide, thioguanine, Thiotepa, TNF-α, Topotecan, Trastuzumab, Tretinoin, Vinblastine, Vincristine, Vindesine, and Vinorelbine.

12. The composition of claim 1, wherein said polysaccharide is a galacto-rhamnogalacturonic based polysaccharide

13. A method of treating a subject with cancer, comprising the administration of a composition having a polysaccharide and one or more chemotherapeutic agents, wherein said chemotherapeutic agents are chemically linked to said polysaccharide.

14. The method of claim 12, wherein said composition is administered to said individual using a method selected from the group consisting of oral, dermal, ophthalmic, sublingual, buccal, intramuscular, intraveneous, intra-arterially, nasal, intraperitoneal, intracranial, intracerebroventricular, intracerebral, intravaginal, intrauterine, rectal, parenteral.

15. The method of claim 13 further comprising an excipient.
